(19) United States
(12) Patent Application Publication
(10) Pub. No.: US 2017/0290774 A1
(43) Pub. Date: Oct. 12, 2017

(54) NEUROKININ-1 RECEPTOR ANTAGONIST COMPOSITION FOR TREATMENT OF DISEASES AND CONDITIONS OF THE RESPIRATORY TRACT

(71) Applicants: PLUS VITECH, S.L., Sevilla (ES); SERVICIO ANDALUZ DE SALUD, Sevilla (ES)

(72) Inventors: Antonio Lara Ruiz, Sevilla (ES); Manuel Vicente Salinas Martín, Sevilla (ES)

(73) Assignees: PLUS VITECH, S.L., Sevilla (ES); SERVICIO ANDALUZ DE SALUD, Sevilla (ES)

(21) Appl. No.: 15/328,825

(22) PCT Filed: Jul. 24, 2015
(86) PCT No.: PCT/EP2015/067006

§ 371 Date: Jan. 24, 2017

Related U.S. Application Data
(60) Provisional application No. 62/028,566, filed on Jul. 24, 2014.

(51) Int. Cl. A61K 9/14; A61K 31/5377; A61K 47/44; A61K 9/16

(52) U.S. Cl. CPC A61K 9/145; A61K 9/16; A61K 31/5377; A61K 47/44; A61K 31/00

(57) ABSTRACT

The present invention relates to a composition comprising a neurokinin-1 receptor antagonist and at least one excipient selected from an oil, a fatty acid or a polyol, as well as to an administration form for administering a formulation, wherein said administration form is selected from: inhalant, nebulization, vapor, smoke or aerosol and comprises said composition. In addition, the invention relates to said composition for use as medicament, said administration form for use as medicament, preferably said composition for use as medicament in the treatment of at least one disease of the respiratory tract, or said administration form for use as medicament in the treatment of at least one disease of the respiratory tract. Moreover, the invention also provides methods for producing said composition and said administration form, as well as a kit of parts comprising said composition and an administration device.

### FIELD OF THE INVENTION

The present invention relates to a composition comprising a neurokinin-1 (NK1) receptor antagonist and at least one excipient selected from an oil, a fatty acid or a polyol. In addition, the present invention also relates to an administration form for administering a formulation comprising said composition. Furthermore, the present invention relates to the use of said composition and administration form as a medicament, particularly for treating diseases and conditions of the respiratory trac90t. Moreover, the invention also provides a method for producing said composition and said administration form, as well as a kit of parts comprising said composition and an administration device.

### BACKGROUND TO THE INVENTION

The present invention relates to a composition for administering a neurokinin-1 (NK1) receptor antagonist. An NK1 receptor antagonist is a type of receptor ligand that inhibits agonist-mediated (including inverse-agonist-mediated) responses upon binding to the NK1 receptor. The NK1 receptor is also known as the tachykinin receptor 1 (TACR1) or substance P receptor (SPR) and is a G protein-coupled receptor found in the central nervous system and peripheral nervous system.

NK1 receptor antagonists possess antidepressant, anxiolytic and antiemetic properties, and serve particular purpose in the prevention of nausea and vomiting associated with cancer chemotherapy.

An example of an NK1 receptor antagonist is aprepitant. Aprepitant (CAS 170729-80-3) is also known by the IUPAC name 5-([(2R,3S)-2-((R)-1-[3,5-bis(trifluoromethyl)phenyl]ethoxy)-3-(4-fluorophenyl)morpholino]methyl)-1H-1,2,4-triazol-3(2H)-one and may be represented by the following structural formula:

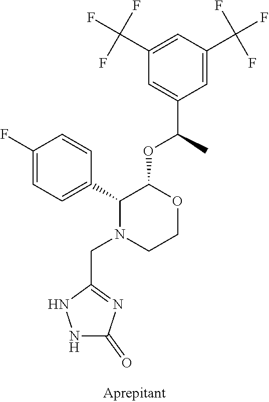 [SMILES: C[C@@H](O[C@@H]1OCCN(Cc2nc(=O)[nH][nH]2)[C@@H]1c1ccc(F)cc1)c1cc(C(F)(F)F)cc(C(F)(F)F)c1]

Aprepitant is an antiemetic compound, whereby said antiemetic effect is brought about by the fact that aprepitant blocks the NK1 receptor. Thus, aprepitant is used in a clinical setting for prevention of acute and delayed chemotherapy-induced nausea and vomiting (CINV) and for prevention of postoperative nausea and vomiting. Aprepitant may also be useful in the treatment of cyclic vomiting syndrome and late-stage chemotherapy-induced vomiting (CIV).

Aprepitant was approved by the FDA in 2003 and in many countries, is marketed as a formulation under the name Emend, wherein said Emend composition comprises, in addition to aprepitant, the following list of excipients:

| Emend capsules | Excipient |
|---|---|
| Capsule contents | Saccarose |
| (comprising aprepitant) | Microcrystalline cellulose (E460) |
| | Hydroxypropylcellulose (E463) |
| | Sodium lauryl sulfate |
| Capsule cover | Gelatine |
| | Titanium dioxide (E171) |
| | Ferric oxide yellow (E172) |

Alternatively, the following composition may be used:

| Tablets | Excipient |
|---|---|
| Tablet contents | Colloidal anhydrous silica |
| (comprising aprepitant) | Sodium lauryl sulfate |
| Tablet cover | Potassium hydroxide |
| | Ferric oxide black (E172) |
| | Lacquer |
| | Printing ink |

The aforementioned Emend composition is that which has been used in order to administer aprepitant orally. The capsule contents comprise individual molecules or particles of aprepitant which are covered with numerous molecules of cellulose which, upon ingestion, dissolve in the aqueous medium of the digestive tract and facilitate dissolution of aprepitant in said aqueous medium, as required for its absorption. In spite of this, the degree of absorption in the digestive tract (oral bioavailability) of the active ingredient is very low, at 67% in a capsule of 80 mg and at 59% in a capsule of 125 mg. In other words, the level of absorption is not only low but is further lowered by increasing the dose, such that higher doses do not increase the absorption in a linear manner, rather they decrease the level of absorption upon increasing the dose.

In this sense, Section 5.2 of the Summary of the Product Characteristics of Emend which is available in Spanish (published as Anexo I: Resumen de las Caracteristicas del Producto) by Merck Sharp & Dohme states that:
“aprepitant exhibits non-linear pharmacokinetics. Not only the clearance but also the absolute bioavailability diminishes upon increasing the dose”.

With respect to the absorption, the average maximum plasma concentration (Cₘₐₓ) of aprepitant is reached after approximately 4 hours (Tₘₐₓ). The oral administration of the capsule with a standard breakfast of approximately 800 Kcal causes an increase in up to 40% of the plasma concentration of aprepitant in the Area Under the Curve (AUC). However, according to the aforementioned Summary of the Product Characteristics of Emend, this increase is not considered of clinical interest.

The pharmacokinetics of aprepitant are non-linear in the interval of the clinical dose. In healthy young adults, the increase in the AUC_{0-∞} was 26% greater in proportion to the dose between the single doses of 80 mg and 125 mg administered in the postprandial state.

After the oral administration of a single dose of 125 mg of Emend on day 1 and 80 mg once a day on days 2 and 3, the AUC_{0-24 h} (average±SD) was 19.6±2.5 micrograms×h/mL and 21.2±6.3 micrograms×h/mL on days 1 and 3, respectively. The Cₘₐₓ was 1.6±0.36 micrograms/mL and 1.4±0.22 micrograms/mL on days 1 and 3, respectively.

Thus, the oral administration of aprepitant not only results in poor absorption but is also inefficient since part of the absorbed active material is eliminated by the portal system upon reaching the liver from the digestive tract in the first hepatic stage when it is metabolised hepatically by cytochrome CYP3A4 (and possibly to a smaller degree with CYP1A2 and CYP2C19), as also stated in Section 5.2 Metabolism (Metabolismo) of the aforementioned Summary of the Product Characteristics, as follows:
“Aprepitant is extensively metabolised. In healthy young adults, aprepitant represents approximately 19% of the plasma radioactivity over 72 hours after a single 100 mg intra venal dose of the propharmaco aprepitant-[C¹⁴], which indicates an important presence of metabolites in the plasma. In human plasma 12 metabolites of aprepitant have been identified. The metabolism of aprepitant is largely produced by oxidation of the morpholine ring and its side-chains, and its resulting metabolites were only weakly active. In vitro studies in which human hepatic microsomes were used indicated that aprepitant is principally metabolized by CYP3A4 and possibly with a lesser contribution through CYP1A2 and CYP2C19.”

Moreover, the oral administration of aprepitant varies with respect to the age, sex, presence in the patient of hepatic or renal insufficiency (wherein said variation may be clinically relevant or that conclusive evidence is not available), as disclosed in Section 5.2 Pharmacokinetics in special populations (Farmacocinética en poblaciones especiales) of the aforementioned Summary of the Product Characteristics, as follows:
“Elderly: After oral administration of a single dose of 125 mg of Emend on day 1 and 80 mg once a day on days 2 and 5 m the AUC_{0-24 h} of aprepitant was 21% greater on day 1 and 36% greater on day 5 in elderly (≧65 years) in comparison with younger adults. The Cₘₐₓ was 10% greater on day 1 and 24% greater on day 5 in elderly in comparison with younger adults. These differences are not considered clinically significant. Emend does not require an altered dose in elderly patients.
Sex: After oral administration of a single dose of 125 mg of Emend, the Cₘₐₓ of aprepitant is 16% greater in women in comparison with men. The half-life of aprepitant is 25% greater in women in comparison with men and its Tₘₐₓ is produced in approximately the same time. These differences are not considered clinically significant. Emend does not require an altered dose as a function of sex.
Pediatric patients: The farmacokinetics of Emend have not been evaluated in patients of less than 18 years of age.
Hepatic insufficiency: Mild hepatic insufficiency (Child-Pugh score of 5 to 6) does not affect to a clinically relevant degree. It is not necessary to adjust the dose in patients with mild hepatic insufficiency. Conclusions related to the influence of moderate hepatic insufficiency (Child-Pugh score of 7 to 8) on the pharmacokinetics of aprepitant cannot be derived from the available data. Clinical or pharmacokinetic data on patients with severe hepatic insufficiency (Child-Pugh score>9) do not exist.

Renal insufficiency: A single dose of 240 mg of Emend was administered to patients with severe renal insufficiency (CrCl<30 ml/min) and to patients with terminal nephropathy which required hemodialysis. In patients with severe renal insufficiency, the AUC_{0-∞} of total aprepitant (non-bonded and bonded to proteins) reduced by 21% and the Cₘₐₓ reduced by 32%, with respect to healthy patients. In patients with terminal nephropathy who had been subject to hemodialysis, the AUC_{0-∞} of total aprepitant reduced by 42% and the Cₘₐₓ reduced by 32%. Due to the modest decreases in the bonding of aprepitant to proteins in patients with renal disease, the AUC of the non-bonded pharmacologically-active drug was not observed to be significantly affected in those patients with renal insufficiency in comparison with healthy subjects. Haemodialysis carried out 4 or 48 hours after the administration did not have significant effects on the pharmacokinetics of aprepitant: less than 0.2% of the dose was recovered from the dialysate.”

Thus, there is a need to provide a means for administering aprepitant which increases the bioavailability and efficiency of this drug, yet minimizes its metabolism, thereby allowing the administered dose to be more tightly controlled depending on the patient group.

### BRIEF DESCRIPTION OF THE INVENTION

The present invention relates to a composition comprising a neurokinin-1 (NK1) receptor antagonist and at least one excipient selected from an oil, a fatty acid or a polyol.

In addition, the present invention relates to an administration form for administering a formulation, wherein said administration form is selected from: inhalant, nebulization, vapor, smoke or aerosol and comprises the composition of the present invention.

Moreover, the present invention relates to a composition for use as medicament or administration form for use as medicament, wherein said composition is defined according to the composition of the present invention and said administration form is defined according to the administration form of the present invention.

Furthermore, the present invention relates to a composition for use as medicament in the treatment of at least one disease of the respiratory tract, or administration form for use as medicament in the treatment of at least one disease of the respiratory tract, wherein said composition is defined according to the composition of the present invention and said administration form is defined according to the administration form of the present invention. In a preferred embodiment of the present invention, said at least one disease of the respiratory tract is selected from cancer, an inflammatory condition and an inflammatory-fibrosis condition.

Analogously, the present invention also relates to a method of treatment of a patient suffering from at least one disease of the respiratory tract which comprises the administration of an effective dose of the composition of the present invention. In a preferred embodiment of the present invention, said method of treatment comprises the administration of an effective dose of the composition of the present invention by using any of the administration forms of the present invention.

In addition, the present invention also relates to a kit of parts for administering a formulation, comprising:

i) a container comprising the composition according the present invention; and
ii) an administration device for administering said composition in any of the administration forms of the present invention from said container.

Moreover, the present invention relates to a method for producing the pharmaceutical composition according to the present invention, which comprises mixing:

a) a neurokinin-1 (NK1) receptor antagonist; and
b) at least one excipient selected from an oil, a fatty acid or a polyol.

Furthermore, the present invention relates to a method for producing the administration form of the present invention, which comprises the method for producing the pharmaceutical composition according to the present invention and subsequently finely-dividing, nebulizing, vaporizing, atomizing and/or aerosolizing said pharmaceutical composition.

### DESCRIPTION OF THE INVENTION

The aforementioned complex problem of the present invention is based on the recognition that the bioavailability and efficiency of aprepitant may be greatly increased by its administration as an inhalable composition comprising aprepitant and at least one excipient selected from an oil, a fatty acid or a polyol. It is assumed that the improved bioavailability and efficiency of said aprepitant composition derives from the hydrophobic nature of the aforementioned excipients which provide it with a high affinity for the lipid membranes of the respiratory tract (such as the surface-active lipoprotein (phospholipoprotein) complex of the pulmonary surfactant), thus allowing aprepitant to be brought into direct contact with said membranes for ready absorption. However, other mechanisms may also play a role in increasing the bioavailability and efficiency of aprepitant. Moreover, compositions comprising aprepitant and at least one excipient selected from an oil, a fatty acid or a polyol are shown to be effective for administration in inhalable administration forms.

In addition to aprepitant, other related neurokinin (NK1) receptor antagonists exist which may also benefit from an improved mode of administration. Said other NK1 receptor antagonists may be used in the present invention. For example, an intravenous prodrug form of aprepitant, fosaprepitant (which has the tradenames Emend Injection and Ivemend) may be used in the present invention.

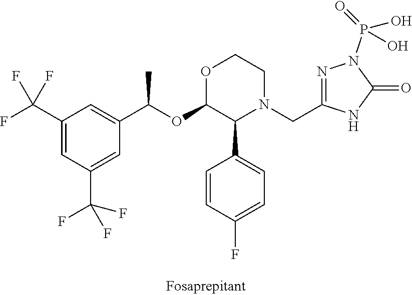 [SMILES: C[C@@H](O[C@H]1OCCN(Cc2nn(P(=O)(O)O)c(=O)[nH]2)[C@H]1c1ccc(F)cc1)c1cc(C(F)(F)F)cc(C(F)(F)F)c1]

Similarly, casopitant (which has the trade names rezonic and zunrisa), maropitant (which has the trade name Cerenia), vofopitant (also known as vofopitant dihydrochloride), L-733,060, lanepitant (also known as LY-303870), vestipitant and L-732,138 are also NK1 receptor antagonists which may be used in the present invention.

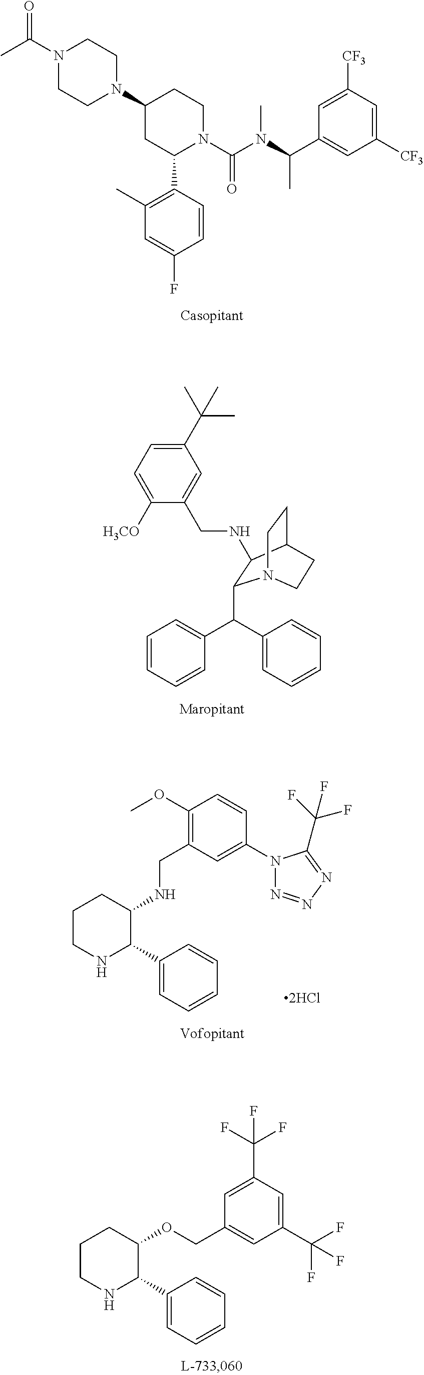 [SMILES: CC(=O)N1CCN([C@H]2CCN(C(=O)N(C)[C@H](C)c3cc(C(F)(F)F)cc(C(F)(F)F)c3)[C@H](c3ccc(F)cc3C)C2)CC1 ; COc1ccc(C(C)(C)C)cc1CNC1C2CCN(CC2)C1C(c1ccccc1)c1ccccc1 ; COc1ccc(-n2nnnc2C(F)(F)F)cc1CN[C@H]1CCCN[C@H]1c1ccccc1 ; FC(F)(F)c1cc(CO[C@H]2CCCN[C@H]2c2ccccc2)cc(C(F)(F)F)c1]

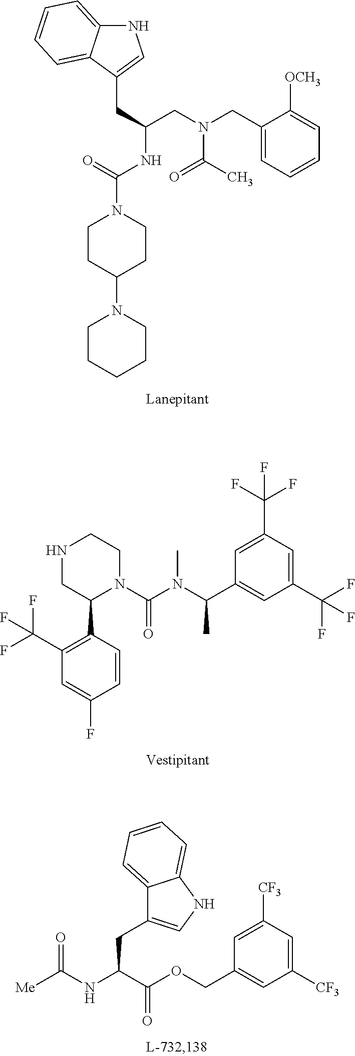 [SMILES: COc1ccccc1CN(C[C@H](Cc1c[nH]c2ccccc12)NC(=O)N1CCC(N2CCCCC2)CC1)C(C)=O ; C[C@H](c1cc(C(F)(F)F)cc(C(F)(F)F)c1)N(C)C(=O)N1CCNC[C@@H]1c1ccc(F)cc1C(F)(F)F ; CC(=O)N[C@@H](Cc1c[nH]c2ccccc12)C(=O)OCc1cc(C(F)(F)F)cc(C(F)(F)F)c1]

Thus, in one embodiment, the present invention relates to a composition comprising a NK1 receptor antagonist selected from aprepitant, fosaprepitant, casopitant, maropitant, vofopitant, L-733,060, lanepitant, vestipitant or L-732,138, and an excipient selected from an oil, a fatty acid or a polyol. In a preferred embodiment of the present invention the NK1 receptor antagonist is selected from aprepitant, fosaprepitant, casopitant, vestipitant or L-732,138, and the excipient is selected from an oil. In a more preferred embodiment of the present invention the NK1 receptor antagonist is selected from aprepitant and fosaprepitant. In a particularly more preferred embodiment, the present invention relates to a composition comprising aprepitant and olive oil, whereby aprepitant is an active compound and olive oil is an excipient.

In the present invention, the concentration of NK1 receptor antagonist in the composition of the invention is between 0.001 mg/mL and 2000 mg/mL, preferably between 1 and 1000 mg/mL, more preferably between 5 and 250 mg/mL.

Olive oil is a fat obtained from plants of the family Oleaceae, in particular plants of the Olea genus, preferably Olea europaea. Preferably, olive oil is a fat obtained from the fruit of said plants, in particular from the olive. Said olive oil may be of extra virgin, virgin, lampante, refined or pomace olive oil quality or grades. As previously stated, one embodiment of the present invention relates to a composition comprising aprepitant and olive oil, preferably virgin olive oil, more preferably extra virgin olive oil.

Since olive oil is composed of the mixed triglyceride esters of oleic acid (55 to 83%), palmitic acid (7.5 to 20%), linoleic acid (3.5 to 21%), stearic acid (0.5 to 5%) and α-linolenic acid (0 to 1.5%), mixed and/or non-mixed triglyceride esters of the aforementioned fatty acids may be used instead of olive oil as an excipient in the composition of the present invention. Moreover, each of the aforementioned fatty acids may also be used instead of olive oil in the present invention.

In this regard, in one embodiment, the composition of the present invention may comprise a NK1 receptor antagonist and mixed and/or non-mixed triglyceride esters of oleic acid, palmitic acid, linoleic acid, stearic acid and α-linolenic acid. In a preferred embodiment, said composition comprises a NK1 receptor antagonist and mixed and/or non-mixed triglyceride esters of oleic acid, linoleic acid and α-linolenic acid, more preferably the triglyceride ester of oleic acid.

Alternatively, the composition of the present invention may comprise a NK1 receptor antagonist and at least one fatty acid. Preferably said fatty acid is selected from oleic acid, palmitic acid, linoleic acid, stearic acid and α-linolenic acid. In a preferred embodiment, said composition comprises a NK1 receptor antagonist and at least one fatty acid selected from oleic acid, linoleic acid and α-linolenic acid, more preferably oleic acid. Thus, in a particularly preferred embodiment, the composition of the invention comprises aprepitant and oleic acid.

Moreover, instead of olive oil, other oils may be used as excipients replacing olive oil in the present invention. Thus, one embodiment of the present invention relates to a composition comprising a NK1 receptor antagonist and an oil, preferably vegetable oil, more preferably wherein said oil is selected from at least one of olive oil, palm oil, soybean oil, rapeseed oil (preferably canola oil), sunflower seed oil, peanut oil, cottonseed oil, palm kernel oil, coconut oil, corn oil, grape seed oil, linseed (flaxseed) oil, rice bran oil, safflower oil, sesame oil, mustard oil, hazelnut oil, walnut oil, almond oil, beech nut oil, cashew oil, macadamia oil, mongongo nut (manketti) oil, pecan oil, pine nut oil, pistachio oil, citrus oils (preferably lemon oil, orange oil or grapefruit seed oil), bitter gourd oil, bottle gourd oil, buffalo gourd oil, butternut squash seed oil, egusi seed oil, pumpkin seed oil, watermelon seed oil, açai oil, black seed oil, blackcurrant seed oil, borage seed oil, evening primrose oil, amaranth oil, apricot oil, apple seed oil, argan oil, avocado oil, babassu oil, ben oil, borneo tallow nut oil, cape chestnut oil, carob pod oil, cocoa butter (theobroma oil), cocklebur oil, cohune oil, coriander seed oil, date seed oil, dika oil, false flax oil, hemp oil, kapok seed oil, kenaf seed oil, lallemantia oil, mafura oil, marula oil, meadowfoam seed oil, niger seed oil, poppyseed oil, nutmeg butter, nutmeg oil, okra seed oil, papaya seed oil, perilla seed oil, persimmon seed oil, pequi oil, pili nut oil, pomegranate seed oil, prune kernel oil, peach kernel oil, quinoa oil, ramtil oil, royle oil, shea butter, sacha inchi oil, sapote oil, seje oil, taramira oil, tea seed (camellia) oil, thistle oil, tigernut (nut-sedge) oil, tobacco seed oil, tomato seed oil, wheat germ oil, neem oil, sea buckthorn oil, Brucea javanica oil, burdock oil, candlenut (kukui nut) oil, carrot seed oil, castor oil, chaulmoogra oil and snowball seed (viburnum) oil.

In a more preferred embodiment, the present invention relates to a composition comprising NK1 receptor antagonist and a vegetable oil, wherein said vegetable oil is selected from at least one of olive oil, palm oil, soybean oil, rapeseed oil, sunflower seed oil, peanut oil, cottonseed oil, palm kernel oil, coconut oil, corn oil, grape seed oil, linseed oil, rice bran oil, safflower oil, sesame oil, mustard oil, hazelnut oil, walnut oil, almond oil, lemon oil, orange oil, evening primrose oil, apricot kernel oil, argan oil, avocado oil, hemp oil, poppyseed oil, perilla seed oil, pomegranate seed oil, tea seed (camellia) oil, wheat germ oil, neem oil, sea buckthorn oil, Brucea javanica oil, castor oil. Furthermore preferably, the present invention relates to a composition comprising a NK1 receptor antagonist, preferably aprepitant, and a vegetable oil, wherein said vegetable oil is selected from at least one of olive oil, palm oil, soybean oil, rapeseed oil, sunflower seed oil, peanut oil, cottonseed oil, palm kernel oil, coconut oil, corn oil, grape seed oil, rice bran oil, sesame oil, walnut oil, evening primrose oil, hemp oil, poppyseed oil, perilla seed oil and neem oil. Most preferably, the present invention relates to a composition comprising a NK1 receptor antagonist, preferably aprepitant, and a vegetable oil, wherein said vegetable oil is selected from at least one of olive oil, palm oil, soybean oil, rapeseed oil, sunflower seed oil, peanut oil, cottonseed oil, palm kernel oil and coconut oil.

Alternatively, the oil comprised in the composition of the present invention may be replaced with a selection of excipients comprising a polyol, more preferably at least one polyol, at least one aroma and water. Preferably the polyol makes up no more than 99% by volume of said composition. Preferably, the at least one polyol is at least one natural polyol derived from plants. More preferably, the at least one polyol comprises a polyether, propylene glycol and/or glycerol (vegetable glycerine). In one embodiment, the olive oil comprised in the composition of the present invention may be replaced with a selection of excipients comprising at least one polyether, glycerol, at least one aroma and water. Preferably the polyether is selected from polyethyleneglycol, polyethylene oxide, polypropylene glycol, polypropylene oxide, paraformaldehyde or polyoxymethylene, more preferably from polyethyleneglycol or polyethylene oxide. Most preferably, the polyether is polyethyleneglycol. In one embodiment of the aforementioned composition, the at least one polyether is present in an amount of <80%, the glycerol is present in an amount of <20%, and the alimentary aromas and water, together with aprepitant (optionally with other active ingredients or excipients) make up the remainder of the composition.

The at least one aroma is a compound or composition that emits an odor which the human brain associates with a particular object or objects. Preferably the at least one aroma is a compound or composition that emits an odor which the human brain associates with tobacco or food, more preferably food, the odor thus being an alimentary odor. Furthermore preferably, the odor is an odor of fruit, apples, pears, strawberries, grapes, oranges, lemons, raspberries, cherries, bananas, peaches, plums, mango, pineapple, coffee, chocolate, cotton candy, cinnamon, vanilla, wintergreen, peppermint, hazelnuts and almonds.

Another aspect of the present invention relates to an administration form for administering a formulation comprising the composition of the present invention. The administration form may be any form that allows the composition of the present invention to be inhaled or forced along the airways of the respiratory tract as a gaseous dispersion of particles, wherein said particles are either in liquid (droplet) or solid (powder, especially micronized powder) form. In one embodiment, the administration form is selected from an inhalant, a nebulization, vapor, smoke or an aerosol. An aerosol may take the form of a spray and an inhalant may take the form of a pulverization. In a preferred embodiment, the administration form is selected from an inhalant, pulverization, spray or aerosol. In a more preferred embodiment, the administration form is selected from an inhalant, a nebulization or an aerosol, more preferably an inhalant or nebulization.

As such, another aspect of the present invention relates to a kit of parts, wherein said kit of parts comprises a container comprising the composition of the present invention, and an administration device for administering said composition in any of the forms of an inhalant, a nebulization, vapor, smoke or an aerosol. Said container may be in the form of a bottle, vial, ampoule, capsule, sachet, syringe or cartridge. Said administration device may be any device which can allow the composition of the present invention to form a fine dispersion in a gas or gas composition at ambient temperature and pressure. Said administration device may use said gas (such as oxygen or tetrafluoroethane) or said gas composition (such as air or an anaesthetic gas composition) at ambient temperature and pressure, or compressed and/or heated forms thereof, to form said administration form. Alternatively, the composition of the present invention or a formulation comprising it may be subjected to heating, combustion, ultrasonication or atomization so as to form a fine dispersion. In a preferred embodiment, the administration device is an inhaler, nebulizer, vaporizer, electronic cigarette, aerosol canister or bottle, atomizer or ventilator.

The administration form of the present invention comprises the composition of the present invention. In addition to comprising the NK1 receptor antagonist and excipient of the composition of the present invention, said administration form may additionally comprise at least one further excipient and/or carrier. Said further excipient and/or carrier may be an inert ingredient or ingredients. For example, the composition of the invention may be mixed with a carrier, diluted by a carrier, enclosed within a carrier, or adsorbed or absorbed on a granular solid carrier.

When the carrier serves as a diluent, it may be solid, semi-solid, or liquid material that acts as a vehicle, excipient, or medium for the active compound. Some examples of suitable carriers are the aforementioned vegetable oils, aforementioned mixed and/or non-mixed triglyceride esters and aforementioned fatty acids, but also include solvents such as alcohols (preferably ethanol), polyethylene glycols, vegetable glycerine, fatty acid monoglycerides (derived from the aforementioned fatty acids), mixed and/or non-mixed fatty acid diglycerides (derived from the aforementioned fatty acids), polyhydroxyethoxylated oils (preferably polyhydroxyethoxylated vegetable oils from the aforementioned vegetable oils, furthermore preferably polyhydroxyethoxylated olive oil), pentaerythritol fatty acid esters, water, and salt solutions such as isotonic saline. Other carriers such as lactose, terra alba, sucrose, cyclodextrin, amylose, magnesium stearate, talc, gelatin, agar, pectin, acacia, lower alkyl ethers of cellulose, silicic acid, fatty acid amines, polyoxyethylene, hydroxymethylcellulose, polyvinylpyrrolidone may also be used.

The formulations may also include wetting agents, emulsifying and suspending agents, preserving agents, sweetening agents, flavouring agents or aromas (preferably alimentary aromas). In addition, the pharmaceutical compositions can be sterilized and mixed, if desired, with auxiliary agents, emulsifiers, salts for influencing osmotic pressure, buffers and/or colouring substances and the like, which do not deleteriously react with the NK1 receptor antagonist of the composition of the invention.

Thus, in one particularly preferred embodiment, the composition of the invention comprises aprepitant and olive oil suitable for administration as an inhaler spray, aerosol or nebulized administration form, using an inhaler or nebulizer as an administration device. In another particularly preferred embodiment, the composition of the invention comprises aprepitant, oleic acid and ethanol, suitable for administration as an inhaler spray, aerosol or nebulized administration form, preferably as an inhaler spray, using an inhaler as an administration device. Alternatively, the composition of the invention comprises aprepitant, polyethyleneglycol (<80%), vegetable glycerine (<20%), alimentary aromas and water, suitable for administration as a vapor administration form using an electronic cigarette as an administration device.

The administration form of the present invention allows the composition of the invention to reach all parts of the respiratory tract in human or animal patients, from the nasal passages to the larynx, pharynx, esophagus, trachae and lung, whereby the locations of the lung which may be reached include the bronchioles and alveoli. It is possible to treat all of the parts of the respiratory tract in the same manner using the composition of the invention.

As such, the composition of the present invention may be used to treat disease or conditions in human or animal patients. Optionally, said composition of the present invention is in one of the administration forms of the present invention. In one embodiment of the present invention, said disease or condition is at least one disease or condition of the respiratory tract. Preferably, said at least one disease or condition of the respiratory tract is a disease or condition which has an associated inflammatory component. In one more preferred embodiment of the present invention, said at least one disease of the respiratory tract is selected from cancer, an inflammatory condition and an inflammatory-fibrosis condition. In an even more preferred embodiment of the present invention, said at least one disease of the respiratory tract is a cancer selected from lung cancer, tracheal cancer, laryngeal cancer, pharyngeal cancer, cancer of the nasal cavities and esophageal cancer. Alternatively, in another even more preferred embodiment of the present invention said at least one disease or condition of the respiratory tract is an inflammatory condition selected from rhinitis, glossitis, buccal aphthous stomatitis, gingivitis, pharyngitis, laryngitis, bronchitis and alveolitis. In a still more preferred embodiment, said bronchitis is selected from allergic bronchitis, asthmatic bronchitis and infectious bronchitis, wherein said infectious bronchitis is preferably bacterial bronchitis or viral bronchitis. In another still more preferred embodiment, said alveolitis is selected from allergic alveolitis, asthmatic alveolitis and infectious alveolitis, wherein said infectious alveolitis is preferably bacterial alveolitis or viral alveolitis. On the other hand, in another even more preferred embodiment of the present invention said at least one disease or condition of the respiratory tract is an inflammatory-fibrosis condition selected from usual interstitial pneumonia, idiopathic interstitial pneumonias and pneumoconiosis.

In a preferred embodiment of the invention, said at least one disease or condition of the respiratory tract is selected from lung cancer, tracheal cancer, laryngeal cancer, pharyngeal cancer, cancer of the nasal cavities, esophageal cancer, rhinitis, glossitis, buccal aphthous stomatitis, gingivitis, pharyngitis, laryngitis, bronchitis, alveolitis, usual interstitial pneumonia, idiopathic interstitial pneumonias and pneumoconiosis.

In addition, the composition of the present invention may be used as an emetic in treating nausea and vomiting associated with cancer chemotherapy in human patients. In particular, the composition of the present invention may be used as an emetic in treating nausea and vomiting associated with cancer chemotherapy used in the treatment of the aforementioned cancers. Preferably, the cancer chemotherapy emesis is cisplatin-induced emesis.

Analogously, the present invention also relates to a method of treatment of a patient suffering from at least one disease of the respiratory tract which comprises the administration of an effective dose of the composition of the present invention. Optionally, said method of treatment is by using any of the administration forms of the present invention. In one preferred embodiment of the foregoing method of treatment, the at least one disease of the respiratory tract is selected from: cancer, an inflammatory condition and an inflammatory-fibrosis condition. In a more preferred embodiment of the method of treatment of the present invention, the at least one disease of the respiratory tract is cancer selected from lung cancer, tracheal cancer, laryngeal cancer, pharyngeal cancer, cancer of the nasal cavities and esophageal cancer. In another more preferred embodiment of the method of treatment of the present invention, the at least one disease of the respiratory tract is an inflammatory condition selected from rhinitis, glossitis, buccal aphthous stomatitis, gingivitis, pharyngitis, laryngitis, bronchitis and alveolitis. In yet another more preferred embodiment of the method of treatment of the present invention, the at least one disease of the respiratory tract is an inflammatory-fibrosis condition selected from usual interstitial pneumonia, idiopathic interstitial pneumonias and pneumoconiosis.

In the present invention, the effective dose of the composition of the present invention is an inhaled dose of between 0.01 and 1.0 mg/kg of bodyweight per day, preferably between 0.05 and 0.5 0 mg/kg of bodyweight per day, more preferably between 0.05 and 0.4 0 mg/kg of bodyweight per day, still more preferably between 0.1 and 0.20 mg/kg of bodyweight per day. Said dose may be distributed into between 1 and 5 sub-doses inhaled at intervals throughout the day, preferably between 2 and 4 subdoses, more preferably 3 subdoses.

The present invention also relates to a method for producing the pharmaceutical composition of the present invention, which comprises mixing an NK1 receptor antagonist according to the foregoing, and olive oil. Optionally said method additionally comprises a step c) of mixing at least one further excipient and/or carrier with said NK1 receptor antagonist and said olive oil. Said method may involve a step of heating, agitation, centrifugation and/or filtration in order to ensure homogeneity of the resulting mixture.

The present invention further relates to a method for producing the administration form according to the present invention, which comprises the foregoing method for producing the pharmaceutical composition according to the present invention and subsequently finely-dividing, nebulizing, vaporizing, atomizing and/or aerosolizing said pharmaceutical composition.

Experimental Results

In the following Examples, aprepitant was used either as a pure compound, as were the other NK1 receptor antagonists, L-732,138, vestipitant and casopitant, or as a component of a commercial formulation (Emend) microencapsulated in cellulose.

### Example 1

i) Pure aprepitant (10 mg) was measured into a vial, to which was added extra virgin olive oil (1 mL). This procedure was repeated in separate vials, each time increasing the amount of aprepitant so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours, and allowed to settle after which time clear solutions were obtained.

ii) Aprepitant microencapsulated in cellulose was measured into a vial such that said vial comprised 10 mg of aprepitant, to which was added extra-virgin olive oil (1 mL). This procedure was repeated in separate vials, each time increasing the amount of aprepitant microencapsulated in cellulose so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours. The resulting suspensions comprising white solids were centrifuged in order to obtain a clear solution.

Aprepitant was found to be completely solubilized in the compositions resulting from i) and ii). Said compositions were each placed (together with approximately 5 mL of air) in syringe barrels. Upon applying pressure to the piston (plunger) of each syringe each of said compositions formed a fine aerosol upon exiting the nozzles thereof.

iii) In order to prove that NK1 receptor antagonists other than aprepitant dissolve in olive oil, the saturation concentration of various NK1 receptor antagonists as solutes in olive oil (solvent) was determined. To this end, increasing quantities of various NK1 receptor antagonists, including aprepitant, as active ingredients were separately mixed in a 10 mL volume of olive oil in order to formulate compositions with a potential range of different concentrations of NK1 receptor antagonists of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. Each of said mixtures was continuously and gently stirred for 10 minutes at ambient temperature (25° C.) and then maintained at rest for 60 minutes. The saturation concentration was considered to be that which was immediately lower in the aforementioned series than a concentration where deposition was observed, but in which no deposition of the active ingredient was observed. The data of the saturation concentration are shown in Table 1.

TABLE 1
Saturation concentrations for different solutes in olive oil

| | Molecular mass | Saturation |
|---|---|---|
| Solute | (g/mol) | concentration (nM) |
| Aprepitant | 534.4 | 60 |
| L-732,138 | 434.4 | 80 |
| Vestipitant | 545.4 | 60 |
| Casopitant | 616.3 | 40 |

iv) In order to prove that the capacity of aprepitant and other NK1 receptor antagonists to dissolve is improved with the addition of an alcohol, the saturation concentration of various NK1 receptor antagonists as solutes in a mixed solvent comprising olive oil was determined. To this end, increasing quantities of various NK1 receptor antagonists, including aprepitant, as active ingredients were separately mixed in a solvent mixture consisting of a 9 mL volume of olive oil and 1 mL of ethanol in order to formulate compositions with a potential range of different concentrations of NK1 receptor antagonists of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. Each of said mixtures was continuously and gently stirred for 10 minutes at ambient temperature (25° C.) and then maintained at rest for 60 minutes. The saturation concentration was considered to be that which was immediately less than a concentration where deposition was observed, but in which no deposition of the active ingredient was observed. The data of the saturation concentrations of different solutes in olive oil:ethanol (9:1) are shown in Table 2.

TABLE 2
Saturation concentrations for different solutes in olive oil:ethanol (9:1)

| | Molecular mass | Saturation |
|---|---|---|
| Solute | (g/mol) | concentration (nM) |
| Aprepitant | 534.4 | 80 |
| L-732,138 | 434.4 | 100 |
| Vestipitant | 545.4 | 80 |
| Casopitant | 616.3 | 65 |

v) In order to prove that oils of types other than olive oil can act as solvents of aprepitant and other NK1 receptor antagonists, the saturation concentration of various NK1 receptor antagonists as solutes in various oils was determined. To this end, increasing quantities of various NK1 receptor antagonists, including aprepitant, as active ingredients were separately mixed in a 10 mL volume of various oils in order to formulate compositions with a potential range of different concentrations of NK1 receptor antagonists of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. Each of said mixtures was continuously and gently stirred for 10 minutes at ambient temperature (25° C.) and then maintained at rest for 60 minutes. The saturation concentration was considered to be that which was immediately less than a concentration where deposition was observed, but in which no deposition of the active ingredient was observed. The data of the maximum concentration of dissolution are shown in Table 3.

TABLE 3
Saturation concentrations for different solutes in different oils

| | Molecular mass | Saturation concentration (nM) | | |
|---|---|---|---|---|
| Solute | (g/mol) | Soy oil | Sunflower oil | Rapeseed oil |
| Aprepitant | 534.4 | 60 | 70 | 80 |
| L-732,138 | 434.4 | 90 | 80 | 90 |
| Vestipitant | 545.4 | 80 | 80 | 70 |
| Casopitant | 616.3 | 60 | 60 | 70 |

### Example 2

The following two compositions were formulated such that the type and amount of excipients comprised therein are analogous to those of the “inhaler spray” denominated “Budesonide Aldo-unión”.

i) Pure aprepitant (10 mg) was measured into a vial, to which was added a solution of oleic acid (0.69 mL) in ethanol (0.31 mL), i.e. a 69% (volume/volume) ethanolic solution of oleic acid. This procedure was repeated in separate vials, each time increasing the amount of aprepitant so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours, after which time clear solutions were obtained.

ii) Aprepitant microencapsulated in cellulose was measured into a vial such that said vial comprised 10 mg of aprepitant, to which was added a solution of oleic acid (0.69 mL) in ethanol (0.31 mL), i.e. a 69% ethanolic solution of oleic acid. This procedure was repeated in separate vials, each time increasing the amount of aprepitant microencapsulated in cellulose so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours. The resulting suspensions comprising white solids were centrifuged in order to obtain a clear solution.

Aprepitant was found to be completely solubilized in the compositions resulting from i) and ii). Said compositions were each placed in a pressurized, metered-dose inhaler and nebulized into a fine aerosol.

### Example 3

i) Pure aprepitant (10 mg) was measured into a vial, to which was added a solution of polyethyleneglycol (0.75 mL), vegetal glycerine (0.15 mL) and apple aroma (0.01 mL) in water (0.09 mL). This procedure was repeated in separate vials, each time increasing the amount of aprepitant so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours, after which time clear solutions were obtained.

ii) Aprepitant microencapsulated in cellulose was measured into a vial such that said vial comprised 10 mg of aprepitant, to which was added a solution of polyethyleneglycol (0.75 mL), vegetal glycerine (0.15 mL) and apple aromas (0.01 mL) in water (0.09 mL). This procedure was repeated in separate vials, each time increasing the amount of aprepitant microencapsulated in cellulose so as to obtain compositions with a potential range of different concentrations of aprepitant of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL and 125 mg/mL. The resulting mixtures were dispersed by gentle agitation at ambient temperature (25° C.) over a time of from 1-24 hours. The resulting suspensions comprising white solids were centrifuged in order to obtain a clear solution.

Aprepitant was found to be completely solubilized in the compositions resulting from i) and ii). Said compositions were each placed in an electronic cigarette and vaporised.

### Example 4

This example demonstrates that the administration of NK1 receptor antagonists is more efficient in the lungs when administered by inhalation of an oil-alcohol mixture in which it is dissolved, than when administered orally. Herein, New Zealand albino rabbits (New Zealand White Rabbits) with an initial average weight of approximately 3 kg were used. The rabbits were fed ad libitum and were monitored daily to check that signs of toxicity had not appeared.

To assess its efficiency on the lungs when administered orally, aprepitant was administered to a group of 3 of said rabbits (group 1) in an oral dose of 10 mg/kg body weight per day, to another group of 3 rabbits (group 2) in an oral dose of 40 mg/kg body weight per day, to a third group (group 3) in an oral dose of 80 mg/kg body weight per day and to a fourth group (group 4) in an oral dose of 100 mg/kg body weight per day.

In parallel, a mixture consisting of aprepitant, as the active ingredient, and olive oil, as a solvent, was prepared. This mixture was administered as an aerosol to the airways of a group of 3 of said rabbits (Group 5) in a dose of 0.05 mg/kg body weight per day, to another group of three rabbits (Group 6) in a dose of 0.1 mg/kg body weight per day, to another group of three rabbits (group 7) in a dose of 0.2 mg/kg body weight per day and to another group of three rabbits (Group 8) in a dose of 0.4 mg/kg body weight per day. The mixture was administered as an aerosol by means of a pediatric inhalation mask connected to a nebulising system with an air compressor, wherein said aerosol and nebulising system had the following characteristics: particle size of 0.5 to 10 micrometres, average particle size 4 micrometres, compressor pressure range 30 to 36 psi (210 to 250 KPa/2.1 to 2.5 bar), operating pressure range 8 to 16 psi (50 to 100 kPa/0.5 to 1.0 bar), flow rate 6 to 8 litres/min.

After seven days of treatment all animals were euthanized and necropsied. Five lung tissue samples were taken from each animal, each corresponding to tissue from the five lung lobes (right superior, right middle, right inferior, left superior and left inferior). Samples were taken at the peripheral level of the lung parenchyma, just below the pleural surface. Tissue samples were also taken from the nasal cavity, pharynx, larynx and bronchi. In all samples the concentration of aprepitant was determined by HPLC.

The concentration of aprepitant measured in lung samples from the rabbits administered orally with aprepitant at doses of 10, 40, 80 and 100 mg/kg per day, is respectively shown in Tables 4 to 7 for Groups 1 to 4. On the other hand, the concentration of aprepitant measured in lung samples from rabbits administered with aprepitant by inhalation at doses of 0.05, 0.1, 0.2 and 0.4 mg/kg per day, is respectively shown in Tables 8 to 11 for Groups 5 to 6. LSPD, LMDP, LIPD, LSPI and LIPI respectively correspond to the concentration of aprepitant measured in the right superior lobe, right middle lobe, right inferior lobe, left superior lobe and left inferior lobe of the lungs in each of the rabbits (cases). The concentrations are expressed in micrograms of aprepitant per gram of lung tissue. The average is the arithmetic average calculated for each row or column of each table. The SD is the standard deviation, calculated for each row or column of each table.

TABLE 4
Lung concentration of orally-administered aprepitant in Group 1.

| | | | | | | Average ± |
|---|---|---|---|---|---|---|
| | LSPD | LMPD | LIPD | LSPI | LIPI | SD |
| Case 1 | 0.4 | 1.4 | 0.9 | 1.0 | 1.4 | 1.02 ± |
| | | | | | | 0.41 |
| Case 2 | 1.2 | 1.5 | 1.0 | 0.5 | 1.1 | 1.06 ± |
| | | | | | | 0.36 |
| Case 3 | 1.1 | 1.1 | 0.6 | 0.5 | 1.5 | 0.96 ± |
| | | | | | | 0.41 |
| Average ± | 0.9 ± | 1.3 ± | 0.8 ± | 0.6 ± | 1.3 ± | |
| SD | 0.4 | 0.2 | 0.2 | 0.2 | 0.2 | |

TABLE 5
Lung concentration of orally-administered aprepitant in Group 2.

| | | | | | | Average ± |
|---|---|---|---|---|---|---|
| | LSPD | LMPD | LIPD | LSPI | LIPI | SD |
| Case 1 | 4.1 | 2.9 | 3.1 | 4.2 | 2.8 | 3.4 ± |
| | | | | | | 0.68 |
| Case 2 | 3.3 | 5.1 | 4.2 | 3.6 | 4.0 | 4.04 ± |
| | | | | | | 0.69 |
| Case 3 | 3.2 | 4.2 | 3.3 | 5.3 | 5.4 | 4.2 ± |
| | | | | | | 1.05 |
| Average ± | 3.5 ± | 4.0 ± | 3.5 ± | 4.3 ± | 4.0 ± | |
| SD | 0.4 | 1.1 | 0.5 | 0.8 | 1.3 | |

TABLE 6
Lung concentration of orally-administered aprepitant in Group 3.

| | | | | | | Average ± |
|---|---|---|---|---|---|---|
| | LSPD | LMPD | LIPD | LSPI | LIPI | SD |
| Case 1 | 4.2 | 5.5 | 6.3 | 5.1 | 3.4 | 4.9 ± |
| | | | | | | 1.13 |
| Case 2 | 3.3 | 5.3 | 5.3 | 5.1 | 4.3 | 4.6 ± |
| | | | | | | 0.86 |
| Case 3 | 4.9 | 4.8 | 5.1 | 3.9 | 3.6 | 4.4 ± |
| | | | | | | 0.67 |
| Average ± | 4.1 ± | 5.2 ± | 5.5 ± | 4.7 ± | 3.7 ± | |
| SD | 0.8 | 0.3 | 0.6 | 0.6 | 0.4 | |

TABLE 7
Lung concentration of orally-administered aprepitant in Group 4.

| | | | | | | Average ± |
|---|---|---|---|---|---|---|
| | LSPD | LMPD | LIPD | LSPI | LIPI | SD |
| Case 1 | 4.8 | 5.1 | 5.9 | 5.2 | 4.8 | 5.16 ± |
| | | | | | | 0.45 |
| Case 2 | 4.3 | 5.4 | 6.1 | 4.5 | 5.1 | 5.08 ± |
| | | | | | | 0.72 |
| Case 3 | 5.1 | 5.8 | 4.9 | 4.3 | 6.1 | 5.24 ± |
| | | | | | | 0.72 |
| Average ± | 4.7 ± | 5.4 ± | 5.6 ± | 4.6 ± | 5.3 ± | |
| SD | 0.4 | 0.3 | 0.6 | 0.4 | 0.6 | |

TABLE 8
Lung concentration of aprepitant administered
by inhalation in Group 5.

| | | | | | | Average ± |
|---|---|---|---|---|---|---|
| | LSPD | LMPD | LIPD | LSPI | LIPI | SD |
| Case 1 | 1.4 | 1.6 | 1.5 | 1.5 | 1.5 | 1.5 ± |
| | | | | | | 0.07 |
| Case 2 | 1.5 | 1.5 | 1.6 | 1.5 | 1.4 | 1.5 ± |
| | | | | | | 0.07 |
| Case 3 | 1.4 | 1.5 | 1.5 | 1.4 | 1.5 | 1.4 ± |
| | | | | | | 0.05 |
| Average ± | 1.4 ± | 1.5 ± | 1.5 ± | 1.4 ± | 1.4 ± | |
| SD | 0.05 | 0.05 | 0.05 | 0.05 | 0.05 | |

TABLE 9
Lung concentration of aprepitant administered
by inhalation in Group 6.

| | | | | | | Average ± |
|---|---|---|---|---|---|---|
| | LSPD | LMPD | LIPD | LSPI | LIPI | SD |
| Case 1 | 2.8 | 2.9 | 2.9 | 2.7 | 2.8 | 2.8 ± |
| | | | | | | 0.08 |
| Case 2 | 2.9 | 2.9 | 2.8 | 2.8 | 2.9 | 2.8 ± |
| | | | | | | 0.05 |
| Case 3 | 2.9 | 2.9 | 2.8 | 2.8 | 2.8 | 2.8 ± |
| | | | | | | 0.05 |
| Average ± | 2.8 ± | 2.9 ± | 2.8 ± | 2.7 ± | 2.8 ± | |
| SD | 0.05 | 0 | 0.05 | 0.05 | 0.05 | |

TABLE 10
Lung concentration of aprepitant administered
by inhalation in Group 7.

| | | | | | | Average ± |
|---|---|---|---|---|---|---|
| | LSPD | LMPD | LIPD | LSPI | LIPI | SD |
| Case 1 | 5.9 | 6.0 | 6.0 | 6.1 | 6.0 | 6.0. ± |
| | | | | | | 0.07 |
| Case 2 | 6.0 | 6.1 | 6.0 | 5.9 | 5.9 | 5.9 ± |
| | | | | | | 0.08 |
| Case 3 | 6.1 | 6.0 | 6.0 | 6.1 | 5.9 | 6.02 ± |
| | | | | | | 0.08 |
| Average ± | 6.0 ± | 6.03 ± | 6.0 ± | 6.03 ± | 5.9 ± | |
| SD | 0.1 | 0.05 | 0 | 0.1 | 0.05 | |

TABLE 11
Lung concentration of aprepitant administered
by inhalation in Group 8.

| | | | | | | Average ± |
|---|---|---|---|---|---|---|
| | LSPD | LMPD | LIPD | LSPI | LIPI | SD |
| Case 1 | 10.0 | 10.1 | 10.4 | 10.2 | 10.1 | 10.1 ± |
| | | | | | | 0.15 |
| Case 2 | 9.8 | 9.9 | 10.0 | 10.0 | 9.9 | 9.9 ± |
| | | | | | | 0.08 |
| Case 3 | 10.1 | 10.0 | 9.9 | 9.9 | 10.0 | 9.9 ± |
| | | | | | | 0.08 |
| Average ± | 9.9 ± | 10.0 ± | 10.1 ± | 10.0 ± | 10.0 ± | |
| SD | 0.1 | 0.1 | 0.2 | 0.1 | 0.1 | |

In conclusion, administration of aprepitant by inhalation is more efficient than oral administration because similar concentrations of this drug may be obtained in the lung at much lower doses when administered by inhalation as opposed to oral administration. In addition, the distribution of aprepitant in the lung is more homogeneous when administered by inhalation rather than orally, as less variability in the measured concentrations is observed between the cases studied and, in each specific case, between different lung lobes. Furthermore, the concentration of aprepitant measured in the lungs is proportional to the dose administered by inhalation, whereas when aprepitant is administered orally the relationship between dose and concentration in lung tissue is non-linear and not proportional. In samples collected from the nasal cavity, larynx, pharynx and bronchi, similar results were obtained, such that administration of aprepitant by inhalation is more efficient (it achieves a concentration of the drug at lower dose), more homogeneous (with less variability between studied subjects) and proportional to the administered dose.

### Example 5

This example demonstrates that aprepitant and other NK1 receptor antagonists which are formulated in an oil-alcoholic solution exhibit greater efficacy in the treatment of respiratory tract diseases such as cancer (e.g. lung, tracheal, laryngeal, pharyngeal, esophageal, nasal cavity cancers, etc.), inflammatory diseases of the respiratory tract (e.g. rhinitis, pharyngitis, laryngitis, bronchitis, etc.), and inflammatory-fibrosis conditions (e.g. interstitial pneumonia, pneumoconiosis, etc.). It is known that the factors TGF-α, TGF-β 1, TGF-β 2, TGF-β 3, SPARC, MMP-3; MMP-7, MMP-9, MMP-11, MMP-13 and MMP-14, TGF-β, NF-kB, EGF, MMP-9, VEGF and TNF-α which are produced by lung cells (pneumocytes, fibroblasts and vascular endothelial cells) and inflammatory cells (macrophages, lymphocytes and polymorphonuclear leukocytes) are involved in the pathophysiology and development of said respiratory tract diseases. In the present example the administration of aprepitant by inhalation is demonstrated to reduce the production of these factors, thus rendering it useful in the treatment of these diseases. In addition, it is demonstrated that administration of aprepitant by inhalation reduces the histological signs of disease. Herein, 27 New Zealand albino rabbits (New Zealand White Rabbit) with an initial average weight about 3 kg were used in this example. All subjects were administered an aerosol of a solution consisting of a mixture of water and sodium hypochlorite (35 g sodium hypochlorite per liter of water) at a rate of 5 mL of said solution by inhalation three times a day for 7 days (days 1 to 7 of experimentation). Subsequently, from days 3 to 7, a group of 3 rabbits (Group 1) was administered an oral dose of 10 mg/kg body weight per day, another group of 3 rabbits (Group 2) was administered an oral dose of 40 mg/kg body weight per day, a third group (Group 3) was administered an oral dose of 80 mg/kg body weight per day and a fourth group (Group 4) was administered an oral dose of 100 mg/kg body weight per day.

In parallel, a mixture consisting of aprepitant as the active ingredient and olive oil as a solvent was prepared. This mixture was administered as an aerosol by inhalation, also from days 3 to 7, to a group of 3 rabbits (group 5) at a dose of 0.05 mg/kg body weight per day, to another group of three rabbits (Group 6) at a dose of 0.1 mg/kg body weight per day, to another group of three rabbits (Group 7) at a dose of 0.2 mg/kg body weight per day and to another group of three rabbits (Group 8) at a dose of 0.4 mg/kg body weight per day. A placebo consisting only of olive oil was administered from days 3 to 7 to a control group of 3 rabbits (placebo group). In parallel, 3 rabbits were kept under identical conditions but were not administered any treatment nor the aforementioned sodium hypochlorite solution (control group). The mixture was administered as an aerosol by means of a pediatric inhalation mask connected to a nebulising system with an air compressor, wherein said aerosol and nebulising system had the following characteristics: particle size of 0.5 to 10 micrometres, average particle size 4 micrometres, compressor pressure range 30 to 36 psi (210 to 250 KPa/2.1 to 2.5 bar), operating pressure range 8 to 16 psi (50 to 100 kPa/0.5 to 1.0 bar), flow rate 6 to 8 litres/min.

On day 7 of the experiment all animals were euthanized and necropsied. Five lung tissue samples were taken from each animal, each corresponding to tissue from the five lung lobes (right superior, right middle, right inferior, left superior and left inferior). Samples were taken at the peripheral level of the lung parenchyma, just below the pleural surface. Tissue samples were also taken from the nasal cavity, pharynx, larynx and bronchi. Immunohistochemistry studies were performed for all samples to evaluate the levels of the markers TGF-α, TGF-β 1, TGF-β 2, TGF-β 3 SPARC, MMP-3; MMP-7, MMP-9, MMP-11, MMP-14, TGF-β, TNF-α and VEGF.

In said immunohistochemistry studies, a sample of each sample was dehydrated by treatment with increasing concentrations of ethanol and finally xylene. Subsequently said dried samples were embedded in paraffin, thus creating a block. Said paraffin blocks were cut on a microtome to a thickness of 5 μm, and the resulting sections (slices) were placed on slides suitable for conducting immunohistochemistry techniques. Subsequently, the sections were deparaffinised by immersion in xylene and then rehydrated through immersion in a series of solutions containing decreasing concentrations of ethanol and, finally, water. Subsequently, these samples were subjected to 10 times atmospheric pressure (10.1 bar) in citrate buffer at pH 6.0, in order to obtain greater exposure to antigens.

The samples were then allowed to cool to room temperature over 10 minutes. Endogenous peroxidase activity was blocked by treatment with 3% hydrogen peroxide over 30 min at room temperature. After washing the samples with 0.05 M Tris buffer, they were incubated with 10% non-immune pig serum over 30 minutes at room temperature. In order to verify the expression of NK1 receptors, the cell samples were incubated in the presence of anti-NK1 antibodies (S8305, Sigma-Aldrich) diluted 1:1000 at 4° C. overnight. After this time they were washed in 0.05M Tris buffer at room temperature. Subsequently, Envision System-HRP (Dako) reagents were added for 30 min at room temperature. Once this time had expired, the samples were again washed with 0.05 M Tris buffer, and immunoreactivity was visualized by light microscopy with a chromogenic solution with 3,3′-diaminobenzidine (DAB+; Dako, USA). In order to differentiate the cell nuclei, these were lightly stained with haematoxylin. Samples that were not incubated with the primary antibody, but wherein this was replaced by a non-immune serum were used as negative controls. In order to evaluate markers in these immunohistochemical assays, specific primary antibodies were used against them at a concentration 1:1000, as detailed in Table 12. Furthermore, for each of the sampled sections were placed on slides and stained with haematoxylin-eosin before subsequently being deparaffinised by immersion in xylene and then rehydrated through immersion in a series of solutions containing decreasing concentrations of ethanol and, finally, immersing them in water, eosin (20 seconds) and hematoxylin (40 seconds). All experiments were performed in sextuplicate. In order to evaluate the degree of immunostaining in each of the six sections, a cell-count was performed in 20 high-power fields (400×) using an Olympus brand microscope (CX31 model). The total number of cells and the number of cells displaying immunostaining were counted in each one of the fields in order to subsequently determine the percentage of cells displaying said immunostaining. In the sections (slices) stained with hematoxylin/eosin the area occupied by fibrosis was assessed.

TABLE 12
Antibodies used in immunohistochemical assays.

| Antibody | Reference code | Supplier | Characteristics |
|---|---|---|---|
| TGFα | SAB4502953 | Sigma-Aldrich | Rabbit polyclonal |
| TGFβ1 | SAB4502954 | Sigma-Aldrich | Rabbit polyclonal |
| TGFβ2 | SAB4502956 | Sigma-Aldrich | Rabbit polyclonal |
| TGFβ3 | SAB4502957 | Sigma-Aldrich | Rabbit polyclonal |
| MMP-7 | SAB4501894 | Sigma-Aldrich | Rabbit polyclonal |
| MMP-9 | SAB4501896 | Sigma-Aldrich | Rabbit polyclonal |
| MMP-11 | SAB4501898 | Sigma-Aldrich | Rabbit polyclonal |
| MMP-14 | SAB4501901 | Sigma-Aldrich | Rabbit polyclonal |
| TNF-α | ab199013 | Abcam | Mouse monoclonal |
| VEGF | ab1316 | Abcam | Mouse monoclonal |

The results obtained show the presence of all the markers which were analysed in the samples derived from the control group and that the NK1 receptor agonist, aprepitant, induces a partial reduction thereof when administered orally. In this regard, the use of non-peptide NK1 receptor antagonists prevent the development and progression of said respiratory tract diseases.

In particular, the results shown in Table 13 show an increase of all the markers analysed in the samples derived from the placebo group, when compared to the control group, and that oral treatment with aprepitant reduces the presence of these markers. However, in Table 14 it can be seen that for groups treated with the NK1 receptor agonist, aprepitant by inhalation, this reduction in the levels of markers is significantly greater, if not complete, despite much lower doses having been administered. In this regard, the use of non-peptide NK1 receptor antagonists by inhalation prevents the development and progression of said respiratory tract diseases. In addition, this reduction is more homogeneous than when administered orally and is more predictable because it is proportional to the dose administered. Furthermore the use of NK1 receptor antagonists by inhalation is more efficient than the oral use of these antagonists, since much lower doses are necessary to achieve the same results.

TABLE 13
Percent increase in marker for the placebo group and Groups
1 to 4 with respect to the control group ± SD in lung tissue.

| | % Med P/C | % Med G1/C | % Med G2/C | % Med G3/C | % Med G4/C |
|---|---|---|---|---|---|
| TGFα | 2,285.12 ± | 1,717.76 ± | 1,308.73 ± | 1,257.29 ± | 1,195.15 ± |
| | 68.71 | 123.54 | 123.54 | 14.95 | 69.13 |
| TGFβ1 | 1,636.51 ± | 1,277.48 ± | 990.28 ± | 858.27 ± | 759.25 ± |
| | 16.26 | 73.42 | 73.42 | 19.26 | 30.90 |
| TGFβ2 | 1,025.49 ± | 775.23 ± | 582.05 ± | 475.25 ± | 438.55 ± |
| | 45.24 | 57.52 | 57.52 | 9.62 | 32.19 |
| TGFβ3 | 1,067.12 ± | 835.84 ± | 655.71 ± | 496.55 ± | 374.47 ± |
| | 6.04 | 54.48 | 54.48 | 28.31 | 10.90 |
| MMP-7 | 455.98 ± | 345.90 ± | 325.50 ± | 220.38 ± | 184.56 ± |
| | 28.24 | 33.38 | 30 | 8.80 | 4.35 |
| MMP-9 | 733.62 ± | 566.19 ± | 384.44 ± | 338.57 ± | 326.02 ± |
| | 103.31 | 24.64 | 24.64 | 59.10 | 17.14 |
| MMP-11 | 633.33 ± | 522.19 ± | 416.18 ± | 339.86 ± | 251.62 ± |
| | 48.27 | 35.99 | 35.99 | 54.57 | 39.25 |
| MMP-14 | 865.63 ± | 683.47 ± | 569.37 ± | 441.85 ± | 354.84 ± |
| | 43.40 | 16.76 | 16.76 | 25.65 | 51.52 |
| TNF-α | 1,355.68 ± | 859.73 ± | 685.01 ± | 607.15 ± | 463.11 ± |
| | 183.07 | 92.78 | 92.78 | 65.62 | 103.08 |
| VEGF | 921.47 ± | 655.77 ± | 512.28 ± | 458.30 ± | 342.93 ± |
| | 72.63 | 56.07 | 56.07 | 45.95 | 15.74 |
| Fibrosis | 1,328.57 ± | 1,272.22 ± | 683.33 ± | 611.11 ± | 516.67 ± |
| | 265.80 | 76.38 | 76.38 | 153.96 | 125.83 |

% Med P/C: percent increase in marker for the group treated with placebo with respect to the control group ± SD;
% Med G1/C: percent increase in marker for the Group 1 with respect to the control group ± SD;
% Med G2/C: percent increase in marker for the Group 2 with respect to the control group ± SD;
% Med G3/C: percent increase in marker for the Group 3 with respect to the control group ± SD;
% Med G4/C: percent increase in marker for the Group 4 with respect to the control group ± SD.

TABLE 14
Percent increase in marker for the placebo group and Groups
5 to 8 with respect to the control group ± SD in lung tissue.

| | % Med P/C | % Med G5/C | % Med G6/C | % Med G7/C | % Med G8/C |
|---|---|---|---|---|---|
| TGFα | 2,285.12 ± | 1,972,41 ± | 937.00 ± | 402.39 ± | 147.11 ± |
| | 68.71 | 32,92 | 8.90 | 6.79 | 7.12 |
| TGFβ1 | 1,636.51 ± | 1,166.88 ± | 492.89 ± | 270.24 ± | 123.01 ± |
| | 16.26 | 28.11 | 7.70 | 3.10 | 1.75 |
| TGFβ2 | 1,025.49 ± | 797.78 ± | 338.44 ± | 198.92 ± | 125.88 ± |
| | 45.24 | 19.23 | 3.19 | 11.96 | 0.30 |
| TGFβ3 | 1,067.12 ± | 775.37 ± | 427.90 ± | 215.59 ± | 169.46 ± |
| | 6.04 | 11.07 | 6.71 | 7.26 | 5.75 |
| MMP-7 | 455.98 ± | 321.48 ± | 232.63 ± | 142.07 ± | 109.05 ± |
| | 28.24 | 14.35 | 11.15 | 6.87 | 6.68 |
| MMP-9 | 733.62 ± | 570.94 ± | 267.29 ± | 183.98 ± | 156.83 ± |
| | 103.31 | 79.44 | 36.80 | 28.86 | 27.94 |
| MMP-11 | 633.33 ± | 520.05 ± | 222.75 ± | 169.57 ± | 128.28 ± |
| | 48.27 | 26.13 | 7.85 | 9.31 | 9.06 |
| MMP-14 | 865.63 ± | 769.70 ± | 323.25 ± | 184.50 ± | 153.36 ± |
| | 43.40 | 49.74 | 25.18 | 15.26 | 6.31 |
| TNF-α | 1,355.68 ± | 980.46 ± | 470.69 ± | 218.38 ± | 151.28 ± |
| | 183.07 | 127.11 | 62.10 | 27.39 | 22.25 |
| VEGF | 921,47 ± | 575.62 ± | 391.91 ± | 200.18 ± | 145.64 ± |
| | 72,63 | 72.93 | 49.18 | 29.19 | 16.94 |
| Fibrosis | 1,328.57 ± | 1,127.78 ± | 433.33 ± | 233.33 ± | 161.11 ± |
| | 265.80 | 265.80 | 115.47 | 28.87 | 34.69 |

% Med P/C: percent increase in marker for the group treated with placebo with respect to the control group ± SD;
% Med G5/C: percent increase in marker for the Group 5 with respect to the control group ± SD;
% Med G6/C: percent increase in marker for the Group 6 with respect to the control group ± SD;
% Med G7/C: percent increase in marker for the Group 7 with respect to the control group ± SD;
% Med G8/C: percent increase in marker for the Group 8 with respect to the control group ± SD.

Similar results were observed in tissue samples obtained from the nasal cavity (cf. Tables 15 and 16), pharynx (cf. Tables 17 and 18), larynx (cf. Tables 19 and 20) and bronchi (cf. Tables 21 and 22).

TABLE 15
Percent increase in marker for the placebo group and Groups 1 to
4 with respect to the control group ± SD in nasal cavity tissue.

| | % Med P/C | % Med G1/C | % Med G2/C | % Med G3/C | % Med G4/C |
|---|---|---|---|---|---|
| TGFα | 3,509.80 ± | 2,824.89 ± | 1,907.98 ± | 1,347.22 ± | 952.16 ± |
| | 88.21 | 194.95 | 342.82 | 145.56 | 41.28 |
| TGFβ1 | 2,773.08 ± | 2,191.33 ± | 1,562.26 ± | 1,491.16 ± | 1,373.96 ± |
| | 32.06 | 144.90 | 168.11 | 66.00 | 41.28 |
| TGFβ2 | 1,940.80 ± | 1,838.85 ± | 1,469.70 ± | 1,290.84 ± | 1,151.85 ± |
| | 74.11 | 69.75 | 262.68 | 88.45 | 27.29 |
| TGFβ3 | 3,187.67 ± | 2,813.99 ± | 2,128.24 ± | 1,451.25 ± | 1,188.89 ± |
| | 389.82 | 342.04 | 618.90 | 88.45 | 107.96 |
| MMP-7 | 2,467.57 ± | 2,267.10 ± | 1,308.16 ± | 1,119.62 ± | 942.01 ± |
| | 478.29 | 544.82 | 288.79 | 88.45 | 291.79 |
| MMP-9 | 1,531.63 ± | 1,355.64 ± | 768.75 ± | 511.58 ± | 328.92 ± |
| | 84.67 | 71.12 | 105.14 | 112.67 | 29,12 |
| MMP-11 | 1,631.45 ± | 1,023.73 ± | 609.60 ± | 392.70 ± | 270.95 ± |
| | 37.29 | 77.89 | 138.77 | 82.34 | 28.58 |
| MMP-14 | 1,422.66 ± | 1,016.51 ± | 680.45 ± | 412.64 ± | 317.68 ± |
| | 89.92 | 67.99 | 81.56 | 82.34 | 38.50 |
| TNF-α | 2,025.60 ± | 1,411.41 ± | 842.93 ± | 658.65 ± | 418.29 ± |
| | 75.38 | 235.76 | 81.56 | 82.34 | 68.03 |
| VEGF | 2,741.12 ± | 1,293.11 ± | 969.58 ± | 716.62 ± | 576.80 ± |
| | 219.07 | 284.53 | 81.56 | 82.34 | 40.97 |
| Fibrosis | 672.73 ± | 513.89 ± | 375.00 ± | 300.00 ± | 300.00 ± |
| | 128.11 | 146.33 | 81.56 | 100.00 | 100.00 |

% Med P/C: percent increase in marker for the group treated with placebo with respect to the control group ± SD;
% Med G1/C: percent increase in marker for the Group 1 with respect to the control group ± SD;
% Med G2/C: percent increase in marker for the Group 2 with respect to the control group ± SD;
% Med G3/C: percent increase in marker for the Group 3 with respect to the control group ± SD;
% Med G4/C: percent increase in marker for the Group 4 with respect to the control group ± SD.

TABLE 16
Percent increase in marker for the placebo group and Groups 5 to
8 with respect to the control group ± SD in nasal cavity tissue.

| | % Med P/C | % Med G5/C | % Med G6/C | % Med G7/C | % Med G8/C |
|---|---|---|---|---|---|
| TGFα | 3,509.80 ± | 2,908.21 ± | 764.46 ± | 495.68 ± | 156.67 ± |
| | 88.21 | 72.84 | 39.44 | 45.61 | 12.98 |
| TGFβ1 | 2,773.08 ± | 2,065.52 ± | 745.49 ± | 480.80 ± | 138.39 ± |
| | 32.06 | 22.42 | 11.90 | 17.71 | 9.96 |
| TGFβ2 | 1,940.80 ± | 1,451.21 ± | 825.63 ± | 252.52 ± | 136.09 ± |
| | 74.11 | 66.04 | 34.06 | 21.33 | 7.57 |
| TGFβ3 | 3,187.67 ± | 2,168.67 ± | 1,051.06 ± | 482.03 ± | 158.62 ± |
| | 389.82 | 266.93 | 34.06 | 108.16 | 15.53 |
| MMP-7 | 2,467.57 ± | 1,835.98 ± | 1,147.87 ± | 444.94 ± | 136.90 ± |
| | 478.29 | 342.42 | 217.18 | 102.68 | 14.90 |
| MMP-9 | 1,531.63 ± | 1,289.77 ± | 588.48 ± | 291.36 ± | 143.08 ± |
| | 84.67 | 42.11 | 34.52 | 24.37 | 12.14 |
| MMP-11 | 1,631.45 ± | 1,215.12 ± | 489.76 ± | 266.47 ± | 133.33 ± |
| | 37.29 | 32.39 | 13.67 | 19.16 | 15.61 |
| MMP-14 | 1,422.66 ± | 1,190.13 ± | 510.81 ± | 206.80 ± | 149.00 ± |
| | 89.92 | 32.39 | 36.50 | 5.96 | 14.70 |
| TNF-α | 2,025.60 ± | 1,495.60 ± | 736.83 ± | 151.81 ± | 122.23 ± |
| | 75.38 | 32.39 | 21.89 | 7.84 | 9.10 |
| VEGF | 2,741.12 ± | 1,727.98 ± | 1,010.56 ± | 323.15 ± | 145.68 ± |
| | 219.07 | 142.31 | 117.65 | 15.27 | 16.82 |
| Fibrosis | 672.73 ± | 700.00 ± | 319.44 ± | 163.89 ± | 147.22 ± |
| | 128.11 | 90.14 | 63.65 | 12.73 | 20.97 |

% Med P/C: percent increase in marker for the group treated with placebo with respect to the control group ± SD;
% Med G5/C: percent increase in marker for the Group 5 with respect to the control group ± SD;
% Med G6/C: percent increase in marker for the Group 6 with respect to the control group ± SD;
% Med G7/C: percent increase in marker for the Group 7 with respect to the control group ± SD;
% Med G8/C: percent increase in marker for the Group 8 with respect to the control group ± SD.

TABLE 17
Percent increase in marker for the placebo group and Groups 1 to
4 with respect to the control group ± SD in pharyngeal tissue.

| | % Med P/C | % Med G1/C | % Med G2/C | % Med G3/C | % Med G4/C |
|---|---|---|---|---|---|
| TGFα | 2,537.60 ± | 2,274.76 ± | 1,907.77 ± | 1,780.45 ± | 1,550.34 ± |
| | 87.23 | 245.50 | 434.67 | 245.67 | 34.23 |
| TGFβ1 | 2,502.27 ± | 2,198.45 ± | 1,562.45 ± | 1,208.45 ± | 1,001.31 ± |
| | 45.36 | 223.43 | 212.34 | 45.56 | 36.23 |
| TGFβ2 | 1,940.80 ± | 1,575.67 ± | 1,235.45 ± | 980.65 ± | 780.54 ± |
| | 74.11 | 58.63 | 262.68 | 67.35 | 34.81 |
| TGFβ3 | 1,245.56 ± | 967.72 ± | 879.34 ± | 659.59 ± | 456.45 ± |
| | 202.78 | 278.34 | 109.34 | 99.67 | 78.45 |
| MMP-7 | 2,324.56 ± | 2,004.34 ± | 1,591.34 ± | 1,456.45 ± | 967.12 ± |
| | 323.32 | 381.48 | 302.56 | 156.51 | 62.78 |
| MMP-9 | 1,356.54 ± | 1,102.28 ± | 945.34 ± | 789.45 ± | 561.21 ± |
| | 76.54 | 65.43 | 125.34 | 109.23 | 89.23 |
| MMP-11 | 1,789.56 ± | 1,387.65 ± | 1,980.45 ± | 980.34 ± | 670.45 ± |
| | 43.25 | 89.45 | 198.45 | 78.37 | 56.67 |
| MMP-14 | 1,324.56 ± | 1,148.43 ± | 980.64 ± | 762.45 ± | 560.45 ± |
| | 67.23 | 45.84 | 67.65 | 78.24 | 56.34 |
| TNF-α | 2,123.55 ± | 1,357.32 ± | 987.63 ± | 789.45 ± | 418.29 ± |
| | 67.43 | 234.43 | 78.76 | 45.35 | 68.03 |
| VEGF | 2,546.45 ± | 1,789.12 ± | 1,256.67 ± | 998.56 ± | 567.45 ± |
| | 178.45 | 299.45 | 78.54 | 67.24 | 34.54 |
| Fibrosis | 605.23 ± | 489.54 ± | 457.45 ± | 380.00 ± | 275.00 ± |
| | 132.22 | 122.44 | 83.45 | 78.00 | 50.00 |

% Med P/C: percent increase in marker for the group treated with placebo with respect to the control group ± SD;
% Med G1/C: percent increase in marker for the Group 1 with respect to the control group ± SD;
% Med G2/C: percent increase in marker for the Group 2 with respect to the control group ± SD;
% Med G3/C: percent increase in marker for the Group 3 with respect to the control group ± SD;
% Med G4/C: percent increase in marker for the Group 4 with respect to the control group ± SD.

TABLE 18
Percent increase in marker for the placebo group and Groups 5 to
8 with respect to the control group ± SD in pharyngeal tissue.

| | % Med P/C | % Med G5/C | % Med G6/C | % Med G7/C | % Med G8/C |
|---|---|---|---|---|---|
| TGFα | 2,537.60 ± | 1,946.43 ± | 1,364.46 ± | 699.78 ± | 101.34 ± |
| | 87.23 | 89.40 | 45.43 | 56.53 | 11.56 |
| TGFβ1 | 2,502.27 ± | 1,804.23 ± | 1,220.34 ± | 609.67 ± | 121.89 ± |
| | 45.36 | 45.34 | 34.54 | 45.34 | 10.79 |
| TGFβ2 | 1,940.80 ± | 1,202.46 ± | 725.63 ± | 252.52 ± | 136.09 ± |
| | 74.11 | 45.34 | 34.06 | 21.33 | 7.57 |
| TGFβ3 | 1,245.56 ± | 879.45 ± | 543.45 ± | 309.45 ± | 112.43 ± |
| | 202.78 | 99.56 | 65.12 | 33.25 | 23.12 |
| MMP-7 | 2,324.56 ± | 1,820.78 ± | 1,109.45 ± | 450.45 ± | 121.80 ± |
| | 323.32 | 298.23 | 112.32 | 99.45 | 10.34 |
| MMP-9 | 1,356.54 ± | 998.67 ± | 505.32 ± | 212.24 ± | 121.23 ± |
| | 76.54 | 43.23 | 24.21 | 17.21 | 10.12 |
| MMP-11 | 1,789.56 ± | 1,324.23 ± | 904.21 ± | 529.34 ± | 109.43 ± |
| | 43.25 | 32.23 | 21.47 | 15.45 | 9.43 |
| MMP-14 | 1,324.56 ± | 1,091.23 ± | 709.21 ± | 245.45 ± | 135.00 ± |
| | 67.23 | 51.12 | 32.50 | 21.20 | 12.42 |
| TNF-α | 2,123.55 ± | 1,532.34 ± | 989.36 ± | 298.67 ± | 167.34 ± |
| | 67.43 | 56.56 | 34.32 | 23.21 | 12.21 |
| VEGF | 2,546.45 ± | 1,858.37 ± | 1,198.56 ± | 654.32 ± | 121.34 ± |
| | 178.45 | 112.25 | 94.32 | 11.32 | 9.45 |
| Fibrosis | 605.23 ± | 480.30 ± | 258.34 ± | 152.23 ± | 128.32 ± |
| | 132.22 | 90.50 | 32.35 | 11.54 | 9.87 |

% Med P/C: percent increase in marker for the group treated with placebo with respect to the control group ± SD;
% Med G5/C: percent increase in marker for the Group 5 with respect to the control group ± SD;
% Med G6/C: percent increase in marker for the Group 6 with respect to the control group ± SD;
% Med G7/C: percent increase in marker for the Group 7 with respect to the control group ± SD;
% Med G8/C: percent increase in marker for the Group 8 with respect to the control group ± SD.

TABLE 19
Percent increase in marker for the placebo group and Groups 1 to
4 with respect to the control group ± SD in laryngeal tissue.

| | % Med P/C | % Med G1/C | % Med G2/C | % Med G3/C | % Med G4/C |
|---|---|---|---|---|---|
| TGFα | 2,450.34 ± | 1,875.56 ± | 1,500.45 ± | 1,320.32 ± | 1,101.28 ± |
| | 56.12 | 321.49 | 248.54 | 145.59 | 87.43 |
| TGFβ1 | 1,958.34 ± | 1,567.67 ± | 1,278.89 ± | 1,067.78 ± | 988.78 ± |
| | 54.32 | 289.56 | 199.67 | 156.89 | 99.87 |
| TGFβ2 | 2,105.45 ± | 1,789.56 ± | 1,398.89 ± | 989.89 ± | 816.89 ± |
| | 88.66 | 390.89 | 270.78 | 178.17 | 120.45 |
| TGFβ3 | 1,545.67 ± | 1,234.56 ± | 1,009.78 ± | 899.98 ± | 567.89 ± |
| | 126.54 | 399.89 | 278.90 | 125.67 | 99.89 |
| MMP-7 | 1,567.45 ± | 1,012.67 ± | 899.78 ± | 788.56 ± | 576.89 ± |
| | 234.34 | 389.78 | 280.78 | 198.78 | 101.89 |
| MMP-9 | 1,367.67 ± | 1,290.89 ± | 983.98 ± | 898.18 ± | 788.78 ± |
| | 78.34 | 432.12 | 348.78 | 198.89 | 99.10 |
| MMP-11 | 987.45 ± | 789.61 ± | 576.17 ± | 456.89 ± | 324.89 ± |
| | 35.27 | 265.28 | 151.89 | 123.89 | 98.45 |
| MMP-14 | 878.35 ± | 678.45 ± | 567.34 ± | 459.43 ± | 387.34 ± |
| | 45.32 | 134.83 | 110.56 | 89.11 | 63.80 |
| TNF-α | 1,234.55 ± | 1,078.45 ± | 809.99 ± | 708.73 ± | 509.87 ± |
| | 54.32 | 289.45 | 178.39 | 123.75 | 98.80 |
| VEGF | 1,467.56 ± | 1,134.34 ± | 980.54 ± | 875.80 ± | 667.82 ± |
| | 98.34 | 239.45 | 128.49 | 112.76 | 93.12 |
| Fibrosis | 500.50 ± | 250.50 ± | 225.70 ± | 200.57 ± | 150.70 ± |
| | 100.00 | 100.30 | 50.50 | 50.10 | 40.50 |

% Med P/C: percent increase in marker for the group treated with placebo with respect to the control group ± SD;
% Med G1/C: percent increase in marker for the Group 1 with respect to the control group ± SD;
% Med G2/C: percent increase in marker for the Group 2 with respect to the control group ± SD;
% Med G3/C: percent increase in marker for the Group 3 with respect to the control group ± SD;
% Med G4/C: percent increase in marker for the Group 4 with respect to the control group ± SD.

TABLE 20
Percent increase in marker for the placebo group and Groups 5 to
8 with respect to the control group ± SD in laryngeal tissue.

| | % Med P/C | % Med G5/C | % Med G6/C | % Med G7/C | % Med G8/C |
|---|---|---|---|---|---|
| TGFα | 2,450.34 ± | 1,820.45 ± | 1,250.74 ± | 783.78 ± | 234.67 ± |
| | 56.12 | 102.34 | 45.80 | 25.74 | 12.23 |
| TGFβ1 | 1,958.34 ± | 1,398.78 ± | 760.87 ± | 320.34 ± | 102.23 ± |
| | 54.32 | 98.78 | 52.76 | 23.63 | 10.60 |
| TGFβ2 | 2,105.45 ± | 1,506.89 ± | 996.89 ± | 560.38 ± | 120.23 ± |
| | 88.66 | 101.45 | 56.49 | 24.12 | 10.90 |
| TGFβ3 | 1,545.67 ± | 1,183.45 ± | 702.33 ± | 305.32 ± | 132.22 ± |
| | 126.54 | 67.39 | 34.12 | 15.12 | 12.56 |
| MMP-7 | 1,567.45 ± | 1,205.45 ± | 809.54 ± | 372.23 ± | 112.25 ± |
| | 234.34 | 34.48 | 23.84 | 14.23 | 9.25 |
| MMP-9 | 1,367.67 ± | 989.34 ± | 567.37 ± | 256.47 ± | 123.23 ± |
| | 78.34 | 43.12 | 23.54 | 12.45 | 24.54 |
| MMP-11 | 987.45 ± | 768.34 ± | 504.34 ± | 203.23 ± | 167.36 ± |
| | 35.27 | 23.67 | 12.32 | 10.56 | 9.50 |
| MMP-14 | 878.35 ± | 639.34 ± | 543.12 ± | 342.23 ± | 201.28 ± |
| | 45.32 | 32.23 | 21.23 | 12.34 | 8.32 |
| TNF-α | 1,234.55 ± | 879.34 ± | 456.34 ± | 323.12 ± | 150.34 ± |
| | 54.32 | 34.12 | 26.27 | 21.23 | 14.23 |
| VEGF | 1,467.56 ± | 950.34 ± | 670.32 ± | 570.12 ± | 250.21 ± |
| | 98.34 | 32.21 | 21.25 | 14.34 | 10.24 |
| Fibrosis | 500.50 ± | 375.50 ± | 275.45 ± | 200.60 ± | 125.40 ± |
| | 100.00 | 100.00 | 50.50 | 20.20 | 10.10 |

% Med P/C: percent increase in marker for the group treated with placebo with respect to the control group ± SD;
% Med G5/C: percent increase in marker for the Group 5 with respect to the control group ± SD;
% Med G6/C: percent increase in marker for the Group 6 with respect to the control group ± SD;
% Med G7/C: percent increase in marker for the Group 7 with respect to the control group ± SD;
% Med G8/C: percent increase in marker for the Group 8 with respect to the control group ± SD.

TABLE 21
Percent increase in marker for the placebo group and Groups 1 to
4 with respect to the control group ± SD in bronchial tissue.

| | % Med P/C | % Med G1/C | % Med G2/C | % Med G3/C | % Med G4/C |
|---|---|---|---|---|---|
| TGFα | 2,345.45 ± | 1,980.45 ± | 1,540.45 ± | 1,190.45 ± | 873.34 ± |
| | 34.12 | 345.23 | 250.45 | 178.12 | 123.34 |
| TGFβ1 | 1,569.34 ± | 1,234.56 ± | 984.45 ± | 768.32 ± | 502.23 ± |
| | 45.67 | 159.34 | 98.67 | 76.35 | 34.21 |
| TGFβ2 | 1,789.56 ± | 1,342.12 ± | 1,008.32 ± | 878.34 ± | 457.45 ± |
| | 34.12 | 112.45 | 88.43 | 78.23 | 45.67 |
| TGFβ3 | 897.45 ± | 672.56 ± | 452.45 ± | 345.32 ± | 236.12 ± |
| | 89.45 | 98.45 | 56.32 | 45.32 | 39.54 |
| MMP-7 | 762.34 ± | 504.23 ± | 406.34 ± | 398.45 ± | 289.56 ± |
| | 34.56 | 83.46 | 58.45 | 45.65 | 32.21 |
| MMP-9 | 984.34 ± | 749.34 ± | 672.94 ± | 495.87 ± | 305.34 ± |
| | 57.34 | 145.43 | 93.92 | 65.45 | 45.82 |
| MMP-11 | 894.56 ± | 784.45 ± | 673.45 ± | 563.45 ± | 453.34 ± |
| | 78.45 | 121.63 | 85.46 | 67.43 | 78.54 |
| MMP-14 | 678.45 ± | 567.32 ± | 434.45 ± | 325.56 ± | 312.56 ± |
| | 45.39 | 132.45 | 89.45 | 43.24 | 56.43 |
| TNF-α | 898.34 ± | 789.45 ± | 580.67 ± | 467.45 ± | 356.34 ± |
| | 34.23 | 201.36 | 145.67 | 112.56 | 89.45 |
| VEGF | 567.23 ± | 457.34 ± | 356.45 ± | 256.34 ± | 203.56 ± |
| | 28.67 | 145.34 | 95.56 | 67.89 | 35.56 |
| Fibrosis | 3,000.00 ± | 2,500.10 ± | 2,000.00 ± | 1,500.00 ± | 1,000.00 ± |
| | 00.00 | 100.50 | 00.00 | 00.00 | 00.00 |

% Med P/C: percent increase in marker for the group treated with placebo with respect to the control group ± SD;
% Med G1/C: percent increase in marker for the Group 1 with respect to the control group ± SD;
% Med G2/C: percent increase in marker for the Group 2 with respect to the control group ± SD;
% Med G3/C: percent increase in marker for the Group 3 with respect to the control group ± SD;
% Med G4/C: percent increase in marker for the Group 4 with respect to the control group ± SD.

TABLE 22
Percent increase in marker for the placebo group and Groups 5 to
8 with respect to the control group ± SD in bronchial tissue.

| | % Med P/C | % Med G5/C | % Med G6/C | % Med G7/C | % Med G8/C |
|---|---|---|---|---|---|
| TGFα | 2,345.45 ± | 1,875.75 ± | 1,267.45 ± | 689.78 ± | 209.45 ± |
| | 34.12 | 60.30 | 38.32 | 38.72 | 12.45 |
| TGFβ1 | 1,569.34 ± | 1,103.34 ± | 708.45 ± | 302.34 ± | 124.37 ± |
| | 45.67 | 23.43 | 16.45 | 12.47 | 9.7 |
| TGFβ2 | 1,789.56 ± | 1,209.34 ± | 878.45 ± | 328.45 ± | 126.45 ± |
| | 34.12 | 76.45 | 32.21 | 15.34 | 10.23 |
| TGFβ | 897.45 ± | 604.34 ± | 439.53 ± | 234.56 ± | 179.19 ± |
| | 89.45 | 102.34 | 50.47 | 43.89 | 23.21 |
| MMP-7 | 762.34 ± | 549.34 ± | 467.45 ± | 356.47 ± | 241.56 ± |
| | 34.56 | 75.45 | 56.45 | 43.21 | 23.24 |
| MMP-9 | 984.34 ± | 673.45 ± | 452.61 ± | 325.54 ± | 153.34 ± |
| | 57.34 | 34.12 | 27.73 | 20.21 | 45.36 |
| MMP-11 | 894.56 ± | 653.23 ± | 438.43 ± | 342.56 ± | 159.42 ± |
| | 78.45 | 67.38 | 51.23 | 34.23 | 34.25 |
| MMP-14 | 678.45 ± | 458.45 ± | 342.32 ± | 246.47 ± | 164.63 ± |
| | 45.39 | 34.31 | 24.32 | 17.28 | 12.53 |
| TNF-α | 898.34 ± | 673.34 ± | 451.23 ± | 325.21 ± | 201.23 ± |
| | 34.23 | 51.36 | 45.32 | 23.21 | 14.24 |
| VEGF | 567.23 ± | 324.53 ± | 235.71 ± | 153.57 ± | 112.71 ± |
| | 28.67 | 43.32 | 24.17 | 16.42 | 8.23 |
| Fibrosis | 3,000.00 ± | 2,000.00 ± | 1,000.00 ± | 500.00 ± | 100.50 ± |
| | 00.00 | 00.00 | 00.00 | 00.00 | 50.50 |

% Med P/C: percent increase in marker for the group treated with placebo with respect to the control group ± SD;
% Med G5/C: percent increase in marker for the Group 5 with respect to the control group ± SD;
% Med G6/C: percent increase in marker for the Group 6 with respect to the control group ± SD;
% Med G7/C: percent increase in marker for the Group 7 with respect to the control group ± SD;
% Med G8/C: percent increase in marker for the Group 8 with respect to the control group ± SD.

1. A composition comprising a neurokinin-1 (NK1) receptor antagonist and at least one excipient selected from an oil, a fatty acid or a polyol.

2. The composition according to claim 1, wherein the neurokinin-1 (NK1) receptor antagonist is aprepitant and the at least one excipient is olive oil.

3. The composition according to claim 1, which is formulated as an administration form selected from inhalant, nebulization, vapor, smoke and aerosol.

4. The composition according to claim 3, wherein said administration form is selected from inhalant, pulverization, spray and aerosol.

5-10. (canceled)

11. A method of treatment of a patient suffering from at least one disease of the respiratory tract which comprises the administration of an effective dose of a composition comprising a neurokinin-1 (NK1) receptor antagonist and at least one excipient selected from an oil, a fatty acid or a polyol.

12. The method of treatment according to claim 11, wherein the composition is formulated as an administration form selected from inhalant, nebulization, vapor, smoke and aerosol.

13. The method of treatment according to claim 11 wherein the at least one disease of the respiratory tract is selected from cancer, an inflammatory condition and an inflammatory-fibrosis condition.

14. The method of treatment according to claim 13 wherein the at least one disease of the respiratory tract is cancer selected from lung cancer, tracheal cancer, laryngeal cancer, pharyngeal cancer, cancer of the nasal cavities and esophageal cancer.

15. The method of treatment according to claim 13 wherein the at least one disease of the respiratory tract is an inflammatory condition selected from rhinitis, glossitis, buccal aphthous stomatitis, gingivitis, pharyngitis, laryngitis, bronchitis and alveolitis.

16. The method of treatment according to claim 13 wherein the at least one disease of the respiratory tract is an inflammatory-fibrosis condition selected from usual interstitial pneumonia, idiopathic interstitial pneumonias and pneumoconiosis.

17. A kit of parts for administering a formulation, comprising:
i) a container comprising a composition comprising a neurokinin-1 (NK1) receptor antagonist and at least one excipient selected from an oil, a fatty acid or a polyol; and
ii) an administration device for administering said composition in an administration form selected from inhalant, nebulization, vapor, smoke and aerosol from said container.

18. The kit of parts according to claim 17, wherein the administration device is an inhaler, nebulizer, vaporizer, electronic cigarette, aerosol canister or bottle, atomizer or ventilator.

19-20. (canceled)

21. The method of treatment according to claim 11, wherein the neurokinin-1 (NK1) receptor antagonist is aprepitant and the at least one excipient is olive oil.

22. The method of treatment according to claim 12, wherein said administration form is selected from inhalant, pulverization, spray and aerosol.

23. The kit of parts according to claim 17, wherein the neurokinin-1 (NK1) receptor antagonist is aprepitant and the at least one excipient is olive oil.

24. The kit of parts according to claim 17, wherein said administration form is selected from inhalant, pulverization, spray and aerosol.
